(19) United States
(12) Patent Application Publication
(10) Pub. No.: US 2013/0323197 A1
(43) Pub. Date: Dec. 5, 2013

(54) IFG-1 DEPENDENT MODULATION OF VSELS

(71) Applicants: Mariusz Ratajczak, Louisville, KY (US); Magdalena Kucia, Louisville, KY (US); Janina Ratajczak, Louisville, KY (US)

(72) Inventors: Mariusz Ratajczak, Louisville, KY (US); Magdalena Kucia, Louisville, KY (US); Janina Ratajczak, Louisville, KY (US)

(73) Assignee: UNIVERSITY OF LOUISVILLE RESEARCH FOUNDATION, INC., Louisville, KY (US)

(21) Appl. No.: 13/877,963

(22) PCT Filed: Oct. 7, 2011
(86) PCT No.: PCT/US11/55473

§ 371 Date: Aug. 19, 2013

Related U.S. Application Data
(60) Provisional application No. 61/390,951, filed on Oct. 7, 2010.
(60) Provisional application No. 61/429,450, filed on Jan. 3, 2011.
(60) Provisional application No. 61/429,394, filed on Jan. 3, 2011.
(60) Provisional application No. 61/432,549, filed on Jan. 13, 2011.

(51) Int. Cl. G01N 33/50; C12Q 1/68

(52) U.S. Cl. CPC G01N 33/5091; C12Q 1/6883
USPC 424/85.1; 435/39; 435/7.21; 514/733; 514/762; 424/93.7; 435/6.11; 426/2

(57) ABSTRACT

Characteristics of the VSELs stem cell population of a mammal are correlated with aging, including VSEL number, imprinting status of genetic loci, and expression of markers of pluripotent stem cells. The present invention provides VSEL-based methods and compositions for determining the biological age of a mammal.

### STATEMENT OF GOVERNMENT INTEREST

This invention was made with Government support under grant numbers DK074720, RR018733, AG031736, and AG032290 awarded by the National Institutes of Health. The Government has certain rights in this invention.

### FIELD OF THE INVENTION

Characteristics of the VSELs stem cell population of a mammal are correlated with aging. The present invention provides methods and compositions for determining the biological age of a mammal.

### BACKGROUND OF THE INVENTION

In all the living species, senescence is an inevitable consequence of life. Several well-known risk factors, such as obesity, diabetes, high calorie consumption and lack of physical activity that lead to atherosclerosis of the cardio-vascular system and cancer, impair the function of vital organs and limit overall life span. As insulin/insulin-like growth factor signaling (IIS) negatively regulates lifespan in worms, flies and mammals, it has been suggested that all these risk factors directly or indirectly affect the aging process, for example, through prolonged insulin/insulin-like growth factor signaling.

### SUMMARY OF THE INVENTION

The pool size and pluripotentiality of VSELs are shown to be correlated with IGF-1 signaling. Further, the number and pluripotentiality of VSELs are shown to provide measures of aging in mammals. Accordingly, the present invention provides a method of determining a mammal's biological age relative to a reference chronological age for the mammal, which comprises obtaining a tissue or blood sample from the mammal, determining the number of VSELs in the tissue or blood sample, comparing the number of VSELs found in the sample to a reference number of VSELs which represents the reference chronological age for mammals of the species, and determining the relative biological age of the subject mammal. Determining the number of VSELs in the test sample or in the individuals of the reference population usually involves contacting the sample with antibodies and separating the VSELs so that they can be isolated and counted or analyzed, or otherwise labeling the cells to allow identification for counting and/or analysis in a mixture of cells in from the sample. In an embodiment of the invention, the mammal is a human.

In certain embodiments, the reference number of VSELs is determined from the number of VSELs in a similar tissue or blood sample from a population of mammals of the same species and chronological age. In other embodiments, the reference number of VSELs is determined from an age-dependent profile for a population of mammals of the same species. In certain embodiments, the number of VSELs from a test subject and from individuals of the reference population is determined as a percent of total nucleated cells.

The invention also provides a method of determining a mammal's biological age relative to a reference chronological age for the mammal, which comprises obtaining a blood or tissue sample from the mammal, determining the methylation state of one or more of the Oct4 promoter, the Igf2-H19 locus, and the RasGRF-1 locus of VSELs in the blood or tissue sample, comparing the methylation state determined in the sample from the mammal to a reference methylation state of one or more of the Oct4 promoter, the Igf2-H19 locus, and the RasGRF-1 locus of VSELs which represents the reference chronological age for mammals of the species, and determining the relative biological age of the subject mammal. Determining the methylation state of one or more of the Oct4 promoter, the Igf2-H19 locus, and the RasGRF-1 locus of VSELs in the test sample or in the individuals of the reference population usually involves contacting the sample with antibodies and isolating the VSELs for analysis. In an embodiment of the invention, the mammal is a human.

The invention also provides a method of determining a mammal's biological age relative to a reference chronological age for the mammal, which comprises obtaining a blood or tissue sample from the mammal, determining the level of expression of one or more of Oct4, Nanog, Sox2, Klf4, and cMyc in VSELs in the tissue or blood sample, comparing the level of expression determined of one or more of Oct4, Nanog, Sox2, Klf4, and cMyc in the VSELs to a reference level of expression of the one or more of Oct4, Nanog, Sox2, Klf4, and cMyc in VSELs which represent the reference chronological age for mammals of the species, and determining the relative biological age of the subject mammal. Determining the level of expression of one or more of Oct4, Nanog, Sox2, Klf4, and cMyc in VSELs in VSELs in the test sample or in the individuals of the reference population usually involves contacting the sample with antibodies and isolating the VSELs for analysis. In an embodiment of the invention, the mammal is a human.

According to the invention, the VSELs that are counted or analyzed are identified as having a preselected pattern of markers. In certain embodiments, the markers expressed on VSELs are identified using antibodies. One set of preselected markers for human VSELs is CD34⁺, lin⁻, CD45⁻. Another set is CD133⁺, lin⁻, CD45⁻. Another set is CXCR4⁺, lin⁻, CD45⁻. In certain embodiments, the set is CD34⁺, lin⁻, CD45⁻ and one or both of CD133⁺and CXCR4⁺.

In certain embodiments, cells identified by the markers set forth above can also be examined for or isolated on the basis of at least one of SSEA-4, Oct-4, Rex-1, and Nanog. In certain embodiments, the VSELs measured in blood samples are characterized by a diameter of 4-5 μm, 4-6 μm, 4-7 μm, 5-6 μm, 5-8 μm, 6-9 μm, or 7-10 μm. In certain embodiments, the VSELs measured in blood or tissue samples possess large nuclei surrounded by a narrow rim of cytoplasm.

In an embodiment of the invention, the VSELs from a peripheral blood sample are measured. In another embodiment, the VSELs from a mobilized peripheral blood sample are measured. In another embodiment, the VSELs from a sample of bone marrow are measured.

In an embodiment in which mobilized VSELs are measured, the method comprises administering an amount of an agent that mobilizes VSELs before obtaining the blood sample. Such agents include, without limitation, G-CSF and GM-CSF.

According to the invention, when the VSELs measured in a test subject (e.g., by numbers of VSELs, methylation status, or gene expression), indicate a biological age greater than the chronological age of the subject, the mammal is treated to reduce insulin/insulin-like growth factor signal transduction. For example, the test subject is treated with a an effective amount of a compound that inhibits signal transduction by one or more of IGF-R, PI3K, mTOR, and AKT. The subject may be treated with a compound that is a biological agent or a chemical agent.

In another embodiment of the invention, VSELs are measured in a test subject to establish a biological age, and the mammal is treated to reduce insulin/insulin-like growth factor signal transduction.

In another embodiment of the invention, VSELs are measured (e.g., by numbers of VSELs, methylation status, or gene expression) in a test subject to which has been administered a compound that inhibits insulin/insulin like growth factor signaling in order to monitor the effect of the treatment on biological age.

In another embodiment, when the VSELs measured in a test subject indicate a biological age greater than the chronological age of the subject, the mammal is treated with an effective amount of resveratrol. In another embodiment, the mammal is treated with lycopene.

In another embodiment, when the number of VSELs determined in a test subject indicates a biological age greater than the chronological age of the subject, the subject is assigned a low calorie diet.

In yet another embodiment, when the VSELs measured in a test subject indicate a biological age greater than the chronological age of the subject, VSELs are administered to the mammal. In a preferred embodiment, the VSELs are autologous VSELs that were collected from the mammal at an earlier time and stored. In a preferred embodiment, the VSELs that are administered express selected markers of pluripotency at a higher level than VSELs of the obtained tissue or blood sample. Markers of pluripotency include, without limitation, one or more of Oct4, Nanog, Sox2, Klf4, and cMyc.

### DESCRIPTION OF THE FIGURES

FIG. 1. Long-living Laron dwarf mice that maintain low levels of IGF-1 have high numbers of VSELs. Panel A: Body weight comparison of Laron dwarf (GHR−/−) and wild type (WT) mice. Panel B: Comparison of mononuclear cells of Laron dwarf and wild type mice in BM. Panel C: Comparison of VSELs, expressed as a proportion of BMNC, between Laron dwarf and wild type mice. Panel D: Comparison of HSCs, expressed as a proportion of BMNC, between Laron dwarf and wild type mice.

FIG. 2. Changes of pluripotentiality genes and genomic imprinting are observed in very small embryonic-like stem cells (VSELs) from young vs old wild-type mice. (a) RQ-PCR analysis of pluripotency (Oct4, Nanog, Sox2, Klf4 and cMyc) in VSELs isolated by FACS from young (4 weeks, 4wk) and old (18 months, 18M) C57BL/6 mice. The relative expression level is represented as the ratio of 18M value to 4wk one and shown as the mean±s.d.; n=4. The DNA methylation level of Oct4 promoter (b) and DMRs for imprinted genes (c). Bisulfite sequencing results of DNA methylation of Oct4 promoter in VSELs isolated from the indicated aged mice. Methylated and unmethylated CpG sites are shown in filled and open circles, respectively. Mean values for percentage of methylated CpG sites in Oct4 promoter (b, n=4) and DMRs (c, n=3) are shown as the mean±s.d.; Student t-test was used for statistic analysis. ^{*}P<0.05, **P<0.01 compared with 4wk. KvDMR, DMR for Kcnq1 locus.

FIG. 3. Laron dwarf mice have increased numbers of VSELs and hematopoietic stem progenitor cells (HSPCs). (a) The analysis of body weight, BM cellularity, VSELs and HSCs ratio between the indicated aged wild-type and Laron dwarf mice. The ratio for Sea-1⁺ Lin⁻ CD45⁻ (VSELs) and Sca-1⁺ Lin⁻ CD45⁺ (HSCs) was evaluated as the number of events per 1×10⁶ BMMNC by using LSR II FACS analyzer. Statistical analysis is done using one way Analysis of Variance (ANOVA) with Bonferroni's Multiple Comparison Test. ^{**}P<0.01 as compared with wild-type counterparts. (b) Bisulfite-sequencing results of DNA methylation of the Oct4 promoter in VSELs isolated from 20-month-old normal heterozygotes (GHR^{+/-}) and Laron dwarf mice (GHR^{-/-}). The numbers indicated below bisulfite-sequencing profiles present the percentage of methylated CpG sites. (c) The number of clonogenic BFU-E, CFU-GM and CFU-Meg from BM MNC isolated from 8- or 20-month-old normal heterozygotic GHR^{+/-} and Laron dwarf GHR^{-/-} mice. Statistical analysis is done using two way ANOVA with Bonferroni post-tests. ^{*}P<0.05, ^{**}P<0.01 as compared with wild-type counterparts.

FIG. 4. Mice with elevated plasma GH and IGF-I levels have reduced numbers of VSELs and HSPCs in BM. Panel A: Decrease in the numbers of VSELs (left) and HSCs (right) in 6 month old and 1 year old bGH transgenice mice. Panel B: Reduced numbers of VSELs (left) and HSCs (right) after injections of wild type mice with porcine GH (pGH). The ratio for Sca-1⁺ Lin⁻ CD45⁻ (VSELs) and Sca-1⁺ Lin⁻ CD45⁺ (HSCs) was evaluated as the number of events per 1×10⁶ BMMNC/body weight by employing LSR II BD FACS analyzer with FACSDiva™ software. Analysis of Variance (ANOVA) with Bonferroni's Multiple Comparison Test *p<0.00001 as compared to normal counterparts.

FIG. 5. Long-lived dwarf mice with low plasma circulating IGF-I levels retain higher number of VSELs in BM during aging. Panel A: Increase in number of VSELs (left) and HSCs (right) in 2 month old and 2 year old Laron dwarf mice (GHR^{-/-} as compared to their normal heterozygote littermates (GHR^{+/-}). Panel B: Increase in number of VSELs (left) and HSCs (right) in 2- and 8 month old Ames dwarf mice (Prop1^{df/df}) as compared to their normal heterozygote littermates (Prop1^{df/+}). The ratio for Sca-1⁺ Lin⁻ CD45⁻ (VSELs) and Sca-1⁺ Lin⁻ CD45⁺ (HSCs) was evaluated as the number of events per 1×10⁶ BMMNC/body weight by employing LSR II BD FACS analyzer with FACSDiva™ software.

Analysis of Variance (ANOVA) with Bonferroni's Multiple Comparison Test *p<0.00001 as compared to normal counterparts.

FIG. 6. GH or IGF-I treatment reduces number of VSELs and HSCs in long lived dwarf mice. Panel A: Reduced numbers of VSELs (left) and HSCs (right) in pGH treated Ames dwarf mice (Prop1^{df/df}). Ames dwarf mice were treated for 6 weeks starting at the age of 2 weeks or 6 months by twice-daily injection of GH (6 μg/g/day). Panel B: Reduced number of VSELs (left panel) and HSCs (right panel) of IGF-I treated Laron dwarf mice (GHR.^{-/-}). Laron dwarf mice were injected twice a day for 3 weeks with recombinant IGF-I. The ratio for Sca-1 ⁺Lin⁻CD45⁻ (VSELs) and Sca-1 ⁺Lin⁻CD45⁺ (HSCs) was evaluated as the number of events per 1×10⁶ BMMNC/body weight by employing LSR II BD FACS analyzer with FACSDiva™ software. Analysis of Variance (ANOVA) with Bonferroni's Multiple Comparison Test *p<0.00001 as compared to normal counterparts.

FIG. 7. The effect of IIS on changes in DNA methylation on Igf2-H19. DMR in VSELs. Bisulfite-sequencing profiles of DNA methylation of DMRs for Igf2-H19 in VSELs isolated from the indicated mice. Methylated and unmethylated CpG sites are shown in filled and open circles, respectively. The numbers under the bisulfite-sequencing profiles indicate the percentage of methylated CpG sites. Panel A. VSELs were FACS-isolated from 6 month (left) and 1 year (right) old bGH transgenice (bGH-TG) mice and their control (bGH-WT) littermates. Panel B. Wild-type (WT) mice were treated with porcine GH at the age of 2 weeks (left) or 6 month (right) for 6 weeks. For control, the same aged mice were treated with saline. Panel C. VSELs were isolated from 2 years old Laron dwarfs (GHR^{-/-}, left) and Ames dwarfs (Prob1^{df/df}, right) and mice and their control heterozygotes (GHR^{+/-}) and Prob1 ^{df/+}) littermates. Panel D. 2 month and 8 month old Ames dwarfs (Prob1^{df/df}) and Panel E-2 month old Laron dwarfs (GHR^{-/-}) were treated with porcine GH or recombinant IGF-I, respectively at the age of 2 weeks or 6 month for 6 weeks. The control same age mice were treated with saline. Panel F. COBRA assay of Igf2-H19 DMR1 (upper-panel) and RasGRF1 DMR (lower-panel) by BstUI restriction enzyme cleavage. The unmethylated DNA (dashed arrow) was not cleaved in contrast to methylated DNA (solid arrow) because of a sequence change in the site recognized by a restriction enzyme after bisulfite reaction. *: non-specific PCR product.

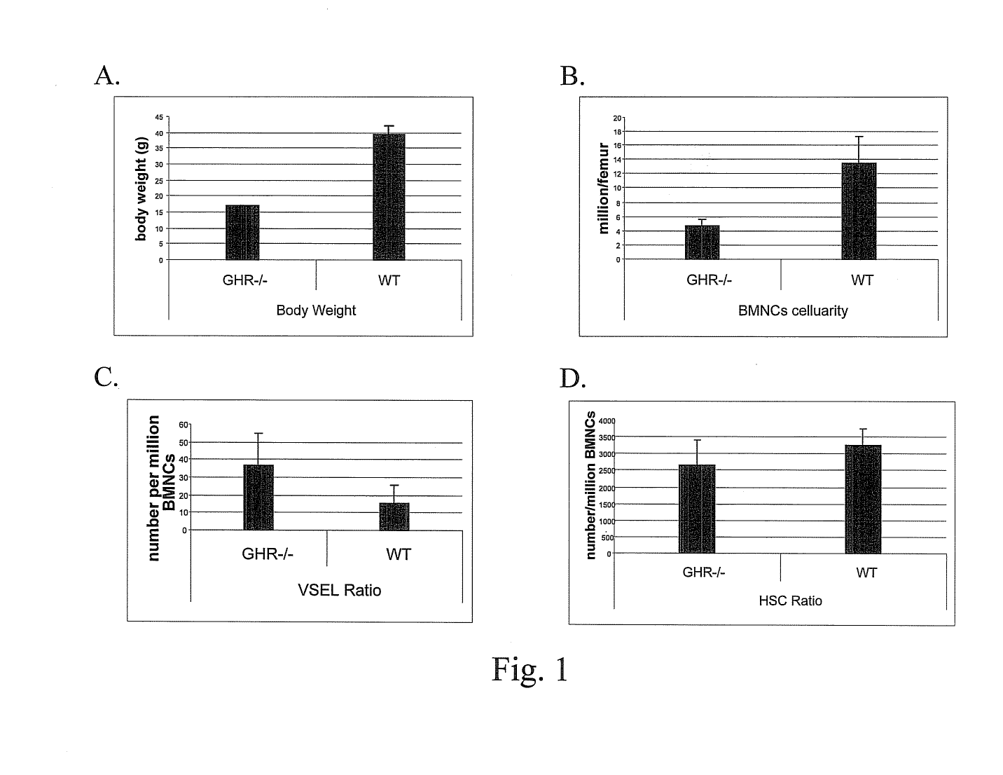

### DETAILED DESCRIPTION

The present invention provides a method for assessing the biological age of a subject. The term “biological age,” as used herein, is a measure of age based on physiological characteristics of a subject which change in an observable manner as the subject ages. The rate of such changes may be affected by, for example, genetics, environment, nutrition, lifestyle, and disease.

According to the invention, the pool size and pluripotentiality of VSELs in mammals, which are shown to be correlated with longevity and IGF-1 signaling, provide a basis for determining biological age. For example, bone marrow (BM) was isolated from Laron dwarf mice are long-living animals. Due to a genetic mutation in the growth hormone receptor (GH-R), Laron dwarf mice maintain very low levels of IGF-1 in peripheral blood (PB). PB cell counts did not reveal any differences in the number of erythrocytes, platelets, and leukocytes between Laron dwarf mice and wild type control littermates, but in contrast, it was observed that Laron dwarf mice have in BM i) ˜3-fold higher number of VSELs in BM tissue, ii) a ˜4-5-fold increase in the number of Sca-1⁺c-kit⁺ lin⁻ (SKL) cells and ii) a >4-fold higher number of clonogenic CFU-GM, BFU-E, and CFU-Meg cells. It is also shown that chronically elevated levels of IGF-1, resulting e.g., from high calorie uptake, are linked to premature depletion of the stem cell pool, including VSELs and HSPCs, and contribute to premature aging.

Age-related VSEL characteristics in mice (and humans) include the following: i) the number of VSELs and their pluripotentiality decreases with age, ii) VSELs from old mice show lower expression of Oct-4, Nanog, Sox2, Klf4, and cMyc, iii) the Oct-4 promoter becomes hypermethylated with age and has a closed chromatin structure, iv) VSELs from old mice show somatic methylation in both Igf2-H19 and Rasgrf1 loci, and v), as a result, VSELs from these mice have increased sensitivity to insulin/insulin factor signaling.

The invention provides a measure of biological age, which is usually expressed with reference to chronological age. Thus, in an embodiment of the invention, one or more age-related VSEL characteristics from a test subject are compared to results taken from a sample population, which serves a reference. As mentioned, the number of circulating VSELs in a subject provides a measure of biological age. Other useful age-related VSEL characteristics include, without limitation, the number of VSELs that can be mobilized from tissue (e.g., bone marrow), gene expression of Oct4, Nanog, Sox2, Klf4, cMyc, or other cellular proteins associated with self renewal and pluripotency, and the methylation state of such genes, including without limitation, methylation of the Oct-4 promoter and Igf2-H19 and Rasgrf1 loci.

In one embodiment, the reference population contains individuals of the same age. A number reflecting the number of VSELs in a sample from an individual is recorded, and a representative value, such as, but not limited to, an average or mean number of VSELs is determined for the reference population. Based on a VSEL value determined for the test subject, the biological age of the test subject is determined to be less that or greater than the reference population.

In another embodiment of the invention, the reference population contains subgroups of individuals of discrete ages. In one non-limiting example, the reference population is made up of discrete ages of 20, 30, 45, 60, and 75 years. In another example, the reference population consists of subjects 10 years apart. As above, a representative value, such as, but not limited to, an average or mean number of VSELs may be determined for each subgroup of such reference populations. The biological age of the test subject may then be expressed as within a range, such as older than 30 and less than 45, or older than 60 and less than 75.

In another embodiment of the invention, the reference population contains individuals of all ages. A linear or non-linear regression analysis of data from the reference population is performed to determine a reference curve, where chronological age of the reference population is the independent variable (e.g., the number of VSELs or expression level of a marker is a function of chronological age). Preferably, a linear or non-linear regression analysis is used to fit the reference curve to the data of the reference population. The observed number of VSELs (or other VSEL marker) from a test subject is then used with the regression curve to extrapolate a biological age for the test subject.

According to the invention, data measured for a test subject into the reference population can optionally be incorporated into the reference database. It may also be desirable to consider other parameters of the reference population, such as, without limitation, sex, race, injury or other medical condition, lifestyle information, and medication, or alternatively, to exclude certain subjects from the reference population on the basis of such parameters. For example, older subjects in the reference population are more likely to be on daily medications than younger subjects. Also, an injury to a subject can lead to VSEL mobilization and higher circulating VSEL numbers. An extreme example is elevated numbers of circulating VSELs in subjects with acute myocardial infarction.

The term “very small embryonic-like stem cell” is also referred to herein as “VSEL stem cell” and refers to pluripotent stem cells. In some embodiments, the VSEL stem cells (“VSELs”) are human VSELs and may be characterized as lin⁻, CD45⁻, and CD34⁺. In some embodiments, the VSELs are human VSELs and may be characterized as lin⁻, CD45⁻, and CD133⁺. In some embodiments, the VSELs are human VSELs and may be characterized as lin⁻, CD45⁻, and CXCR4⁺. In some embodiments, the VSELs are human VSELs and may be characterized as lin⁻, CD45⁻, CXCR4⁺, CD133⁺, and CD34⁺. In some embodiments VSELs express at least one of SSEA-4, Oct-4, Rex-1, and Nanog, and possess large nuclei surrounded by a narrow rim of cytoplasm, and contain embryonic-type unorganized chromatin. VSELs also have high telomerase activity. In some embodiments, the VSELs are human VSELs and may be characterized as lin⁻, CD45⁻, CXCR4⁺, CD133⁺, Oct 4+, SSEA4⁺, and CD34^{±}. In some embodiments, the human VSELs may be less primitive and may be characterized as lin⁻, CD45⁻, CXCR4⁺, CD133⁻, and CD34⁺. In some embodiments, the human VSELs may be enriched for pluripotent embryonic transcription factors, e.g., Oct-4, Sox2, and Nanog. In some embodiments, the human VSELs may have a diameter of 4-5 μm, 4-6 μm, 4-7 μm, 5-6 μm, 5-8 μm, 6-9 μm, or 7-10 μm.

In some embodiments, the VSELs are murine VSELs characterized by lin⁻, CD45⁻, Sca-1^{±}. In other embodiment, the VSELs are murine VSELs characterized by lin, CD45, CXCR4⁺. In other embodiment, the VSELs are murine VSELs characterized by lin, CD45⁻, Sca-1^{±}, CXCR4⁺. In other embodiments, the VSELs are murine VSELs characterized by expression of one or more of SSEA-1, Oct4, Nanog, and Rex-1. In some embodiments, the murine VSELs have a diameter of 3-5 μm, 4-5 μm, or 4-6 μm.

Detection, counting, and/or isolation of VSELs for further analysis usually involves antibodies. Examples of antibodies used for VSEL analysis include, without limitation, those that bind to lineage markers, CD45, CD34, CD133, and CXCR4.

The term “CD45” refers to a tyrosine phosphatase, also known as the leukocyte common antigen (LCA), and having the gene symbol PTPRC. This corresponds to GenBank Accession Nos. NP_{—}002829 (human), NP_{—}035340 (mouse), NP_{—}612516 (rat), XP_{—}002829 (dog), XP_{—}599431 (cow) and AAR16420 (pig). The amino acid sequences of additional CD45 homologs are also present in the GenBank database, including those from several fish species and several non-human primates. “CD34” refers to a cell surface marker found on certain hematopoietic and non-hematopoietic stem cells, and having the gene symbol CD34. The GenBank database discloses amino acid and nucleic acid sequences of CD34 from humans (e.g., AAB25223), mice (NP_{—}598415), rats (XP_{—}223083), cats (NP_{—}001009318), pigs (MP_{—}999251), cows (NP_{—}776434), and others. In mice, some stem cells also express the stem cell antigen Sca-1 (GenBank Accession No. NPJ334868), also referred to as Lymphocyte antigen Ly-6A.2.

Thus, the subpopulation of CD45⁻ stem cells represents a subpopulation of all CD45⁻ cells that are present in the population of cells prior to the separating step. Thus, in some embodiments, the subpopulation of CD45⁻ stem cells from a human are CD34⁺/CXCR4⁺/lin⁻/CD45⁻. In some embodiments, the subpopulation of CD45⁻ stem cells are from a mouse are Sca-1⁺/lin⁻/CD45⁻.

The isolation of the disclosed subpopulations can be performed using any methodology that can separate cells based on expression or lack of expression of the one or more of the CD45, CXCR4, CD34, AC133, Sca-1, CD45R/B220, Gr-1, TCRαβ, TCRγδ, CD11b, and Ter-119 markers including, but not limited to fluorescence-activated cell sorting (FACS).

As used herein, lin⁻ refers to a cell that does not express any of the following markers: CD45R/B220, Gr-1 , TCRαβ, TCRγδ, CD11b, and Ter-119. These markers are found on cells of the B cell lineage from early Pro-B to mature B cells (CD45R/B220); cells of the myeloid lineage such as monocytes during development in the bone marrow, bone marrow granulocytes, and peripheral neutrophils (Gr-1); thymocytes, peripheral T cells, and intestinal intraepithelial lymphocytes (TCRαβ and TCRγδ); myeloid cells, NK cells, some activated lymphocytes, macrophages, granulocytes, B1 cells, and a subset of dendritic cells (CD11b); and mature erythrocytes and erythroid precursor cells (Ter-119).

Separation steps can be performed in a stepwise manner as a series of steps or concurrently. For example, the presence or absence of each marker can be assessed individually, producing two subpopulations at each step based on whether the individual marker is present. Thereafter, the subpopulation of interest can be selected and further divided based on the presence or absence of the next marker.

Alternatively, the subpopulation can be generated by separating out only those cells that have a particular marker profile, wherein the phrase “marker profile” refers to a summary of the presence or absence of two or more markers. For example, a mixed population of cells can contain both CD34⁺ and CD34⁻ cells. Similarly, the same mixed population of cells can contain both CD45⁺ and CD45⁻ cells. Thus, certain of these cells will be CD34⁺/CD45⁺, others will be CD34⁺/CD45⁻, others will be CD34⁻/CD45⁺, and others will be CD34⁻/CD45-. Each of these individual combinations of markers represents a different marker profile. As additional markers are added, the profiles can become more complex and correspond to a smaller and smaller percentage of the original mixed population of cells. In some embodiments, the cells of the presently disclosed subject matter have a marker profile of CD34⁺/CXCR4⁺/lin⁻/CD45⁻, and in some embodiments, the cells of the presently disclosed subject matter have a marker profile of Sca-1⁺/lin⁻/CD45⁻. In some embodiments of the presently disclosed subject matter, antibodies specific for markers expressed by a ceil type of interest (e.g., polypeptides expressed on the surface of a CD34⁺/CXCR4⁺lin⁻/CD45⁻ or a Sca-1⁺/lin⁻/CD45-cell) are employed for isolation and/or purification of subpopulations cells that have marker profiles of interest. It is understood that based on the marker profile of interest, the antibodies can be used to positively or negatively select fractions of a population, which in some embodiments are then further fractionated.

In some embodiments, a plurality of antibodies, antibody derivatives, and/or antibody fragments with different specificities is employed. In some embodiments, each antibody, or fragment or derivative thereof, is specific for a marker selected from the group including but not limited to Ly-6A/E (Sca-1), CD34, CXCR4, AC133, CD45, CD45R, B220, Gr-1, TCRαβ, TCRγδ, CD11b, Ter-119, c-met, LIF-R, SSEA-1, Oct-4, Rev-1, and Nanog. In some embodiments, cells that express one or more genes selected from the group including but not limited to SSEA-1, Oct-4, Rev-1, and Nanog are isolated and/or purified.

In some embodiments, each antibody, or fragment or derivative thereof, comprises a detectable label. Different antibodies, or fragments or derivatives thereof, which bind to different markers can comprise different detectable labels or can employ the same detectable label. A variety of detectable labels are known to the skilled artisan, as are methods for conjugating the detectable labels to biomolecules such as antibodies and fragments and/or derivatives thereof. As used herein, the phrase “detectable label” refers to any moiety that can be added to an antibody, or a fragment or derivative thereof, that allows for the detection of the antibody.

Representative detectable moieties include, but are not limited to, covalently attached chromophores, fluorescent moieties, enzymes, antigens, groups with specific reactivity, chemiluminescent moieties, and electrochemically detectable moieties, etc. In some embodiments, the antibodies are biotinylated. In some embodiments, the biotinylated antibodies are detected using a secondary antibody that comprises an avidin or streptavidin group and is also conjugated to a fluorescent label including, but not limited to Cy3, Cy5, and Cy7. In some embodiments, the antibody, fragment, or derivative thereof is directly labeled with a fluorescent label such as Cy3, Cy5, or Cy7. In some embodiments, the antibodies comprise biotin-conjugated rat anti-mouse Ly-6A/E (Sca-1; clone E13-161.7), streptavidin-PE-Cy5 conjugate, anti-CD45-APCCy7 (clone 30-F11), anti-CD45R/B220-PE (clone RA3-6B2), anti-GM-PE (clone RB6-8C5), anti-TCRαβ-PE (clone H57-597), anti-TCRγδ PE (clone GL3), anti-CD11b PE (clone M1/70) and anti-Ter-119-PE (clone TER-119). In some embodiments, the antibody, fragment, or derivative thereof is directly labeled with a fluorescent label and cells that bind to the antibody are separated by fluorescence-activated cell sorting (FACS). Additional detection strategies are known to the skilled artisan.

While FACS scanning is a convenient method for purifying subpopulations of cells, it is understood that other methods can also be employed. An exemplary method that can be used is to employ antibodies that specifically bind to one or more of CD45, CXCR4, CD34, AC133, Sca-1, CD45R/B220, GM 1 TCRαβ, TCRγδ, CD11b, and Ter-119, with the antibodies comprising a moiety (e.g., biotin) for which a high affinity binding reagent is available (e.g., avidin or streptavidin). For example, a biotin moiety could be attached to antibodies for each marker for which the presence on the cell surface is desirable (e.g., CD34, Sca-1, CXCR4), and the cell population with bound antibodies could be contacted with an affinity reagent comprising an avidin or streptavidin moiety (e.g., a column comprising avidin or streptavidin). Those cells that bound to the column would be recovered and further fractionated as desired. Alternatively, the antibodies that bind to markers present on those cells in the population that are to be removed (e.g., CD45R/B220, Gr-1, TCRαβ, TCRγδ, CD11 b, and TeM 19) can be labeled with biotin, and the cells that do not bind to the affinity reagent can be recovered and purified further. It is also understood that different separation techniques (e.g., affinity purification and FACS) can be employed together at one or more steps of the purification process.

Peripheral blood can be tested directly, for example by counting VSELs in a peripheral blood sample. In certain embodiments, it may be desirable to mobilize VSELs in the peripheral blood from bone marrow and other tissues prior to collection of a blood sample. The term “mobilization” as used herein refers to the process whereby cells leave the bone marrow or other organs and enter the blood. Mobilization may be effectuated by a combination of chemoattractants (e.g. cytokines) and loss of adhesiveness of pools or populations of stem cells residing in stem cell niches in peripheral tissues and the bone marrow.

A mobilizing agent may be administered prior to collection of peripheral blood which will increase the number of VSELs present. In some embodiments, the VSEL mobilizing agent is G-CSF or GM-CSF. In some embodiments, other mobilizing agents can be administered to a subject including, but not limited to, Flt-3 ligand, stem cell factor (SCF), dexamethazone, a CXCR4 receptors inhibitor, Interleukin-1 (IL-1), Interleukin-3 (IL-3), Diniplestim (IL-3 agonist), Leridistim (IL-3 agonist-G-CSF chimeric molecule), Progenipoietin-1 (Flt-3 ligand-G-CSF chimeric molecule), peg-filgrastim (NEULASTA™), Interleukin-8 (IL-8), PIXY-321 (GM-CSF/IL-3 fusion protein), macrophage inflammatory protein, stem cell factor (SCF), and thrombopoietin. Other mobilizing agents include, without limitation, CXCR4 inhibitors, such as AMD3100, ALX40-4C, T22, T134, T140 and TAK-779. One or more doses of the mobilizing agent is administered to the test subject prior to collection. In certain embodiments, 1, 2, 3, or more doses administered. Preferably, the doses are administered once or twice per day, followed by collection. In preferred embodiments, one or two doses are administered. Additional doses may further increase the number of VSELs, but at the expense of convenience for the subject. Preferably, test samples and data used to construct a reference database are obtained from subjects that have undergone the same mobilization protocol.

VSEL pluripotency decreases during aging. VSELs from young mice show erasure of differentially methylated regions (DMRs) for Igf-2-H19 and RasGrf1 loci and thus do not express IGF-2 and RasGRF1. In contrast, VSELs from old mice show the somatic type of methylation at both Igf2-H19 and RasGrf1 loci, which increases expression of Igf-2 and RasGRF1 and, thus, their sensitivity to insulin factor signaling. This indicates chronic IIS via RasGRF1 may contribute to age-related depletion of VSELs and senescence. The increase in methylation at these loci during aging corresponds to the parallel reduction in expression of the pluripotency master-regulators, such as Oct4, Nanog, Sox2, Klf4, and cMyc. At the molecular level, the Oct4 promoter in VSELs becomes gradually hypermethylated with age.

In some embodiments, the present invention provides a method of determining a mammal's biological age relative to a reference chronological sequence which comprises determining the methylation state of the Oct4 promoter, or Igf2-H19, or RasGRF1.

As used herein, the term “imprinted” and grammatical variants thereof refers to a genetic locus for which one of the parental alleles is repressed and the other one is transcribed and expressed, and the repression or expression of the allele depends on whether the genetic locus was maternally or paternally inherited. Thus, an imprinted genetic locus can be characterized by parent-of-origin dependent monoallelic expression: the two alleles present in an individual are subject to a mechanism of transcriptional regulation that is dependent on which parent transmitted the allele. Imprinting can be species- and tissue-specific as well as a developmental-stage-specific phenomenon (see e.g., Weber et al. (2001) Mech Devel 101:133-141; Murphy & Jirtle (2003) Bioessays 25:577-588).

At least 80 loci have been found to be imprinted in mammals (see Morison et al. (2005) Trends Genet 21:457-465). As disclosed herein, several of these loci have been found to be differentially imprinted in VSELs versus other stem cell types. These loci include, but are not limited to the Igf2/H19 locus, the Rasgrf1 locus, the Igf2R locus, the Kcnq1 locus, Peg1/Mest locus, the Meg3 locus, the p57^{KIP2} locus, the p21^{CIP1} locus, the p18^{INK4c} locus, and the SNRPN locus.

As used herein, the term “Igf2” refers to insulin-like growth factor 2 (somatomedin A), which corresponds to GenBank Accession Nos. NC_{—}000011 (genomic sequence from human chromosome 11, nucleotides 2,150,347 to 2,170,833), NM_{—}000612 (transcript variant 1 cDNA sequence), and NP_{—}000603.1 (amino acid sequence encoded by the transcript variant 1 cDNA sequence). The Igf2 locus has been shown to be imprinted, with the maternal allele being methylated (see Kobayashi et al. (2006) Genome Res 113:130-137).

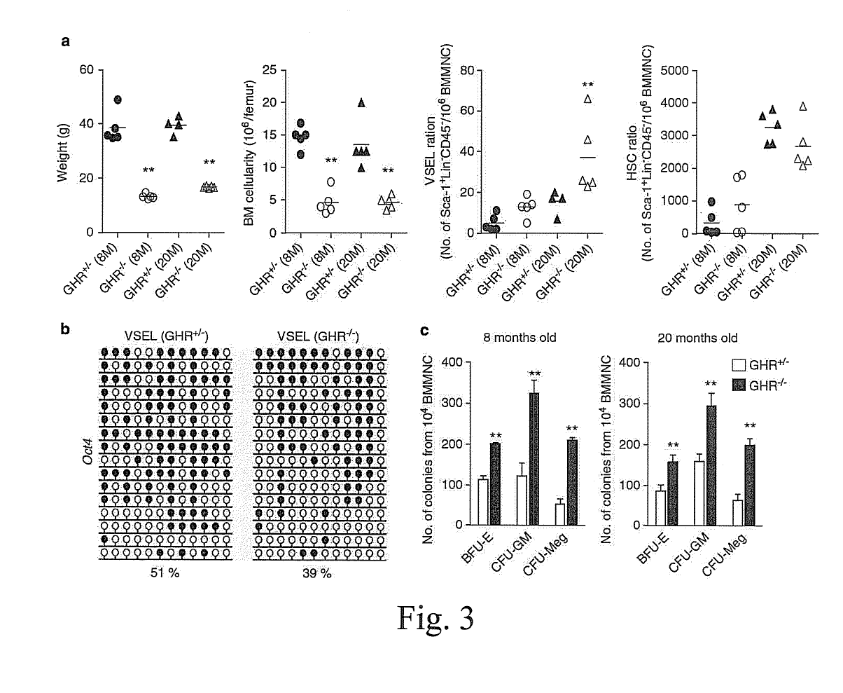

As used herein, the term “H19” refers to H19, which is an imprinted, maternally-expressed but non-protein coding RNA that corresponds to GenBank Accession Nos. NC 000011 (genomic sequence from human chromosome 11, nucleotides 2,016,406 to 2,019,065) and NR_{—}002196 (cDNA sequence). The H19 locus is located on human chromosome 11 in the vicinity of the insulin-like growth factor 2 (IGF2) locus. The H19 locus is expressed from the maternally-inherited chromosome, whereas the Igf2 locus is expressed from the paternally-inherited chromosome. There is a differentially-methylated region (DMR) referred to as “DMR1” located between the promoters for Igf2 and H19 (see FIG. 3A), and as disclosed herein, methylation differences between VSELs and other cell types were identified at DMR1.

As used herein, the term “Rasgrf1” refers to Ras protein-specific guanine nucleotide-releasing factor 1. This locus corresponds to GenBank Accession Nos. NC 000015 (genomic sequence from human chromosome 15, nucleotides 79,252,289 to 79,383,215), NM 002891 (nucleotide sequence of the transcript variant 1 cDNA), and NP 002882 (amino acid sequence encoded by NM_{—}002891). The Rasgrf1 locus has been shown to be imprinted by paternal allele methylation at a DMR located 30 kilbase pairs 5′ of its promoter (Yoon et al. (2005) Mol Cell Biol 25:11184-11190).

As used herein, the term “Igf2R” refers to the insulin-like growth factor 2 receptor, the locus for which corresponds to GenBank Accession Nos. NC_{—}000006 (genomic sequence from human chromosome 6, nucleotides 160,390,131 to 160,527,583), NM_{—}000876 (nucleotide sequence of a cDNA derived from this locus), and NP_{—}000867 (amino acid sequence encoded by NM_{—}000876). The Igfr2 locus has been shown to be imprinted, wherein in most tissues, expression from the paternal allele is suppressed by methylation while the maternal allele is completely unmethylated and expressed. In the central nervous system, however, both parental alleles are unmethylated and expressed (see Hu et al. (1998) Mol Endocrinol 12:220-232).

As used herein, the term “Kcnq1” refers to potassium voltage-gated channel, KQT-like subfamily, member 1, the locus for which corresponds to GenBank Accession Nos. NC_{—}000011 (genomic sequence from human chromosome 11, nucleotides 2,466,221 to 2,870,340), NM_{—}000218 (transcript variant 1 cDNA sequence), and NP_{—}000209 (amino acid sequence encoded by NM_{—}000218). An imprint control region (ICR) has been identified in intron 10 of the human Kcnq1 gene (Thakur et al. (2004) Mol Cell Biol 24:7855-7862).

Various tests that are known to one of ordinary skill in the art can identify and/or assay for the imprinting status of these and other imprinted genes. For example, methylation profiles (i.e., a summary of the methylation status of one or more loci in a cell or cell type) can be detected by simple hybridization analysis (e.g., Southern blotting) of nucleic acids cleaved with methyl-sensitive or methyl-dependent restriction endonucleases to detect methylation patterns. Typically, these methods involve use of one or more targets that hybridize to at least one sequence that may be methylated. The presence or absence of methylation of a restriction sequence is determined by the length of the polynucleotide hybridizing to the probe. This and other methods for detecting DNA methylation are described in, e.g., Thomassin et al. (1999) Methods 19:465-475 and U.S. Pat. No. 7,186,512.

One such method is bisulfite sequencing (see also Warnecke et al. (1990) Genomics 51:182-190). The phrase “bisulfite sequencing” refers to the use of bisulfite to modify DNA following by sequencing of the modified DNA to determine the methylation pattern of the (unmodified) DNA. Bisulfite sequencing takes advantage of the addition of a methyl group to the carbon-5 position of cytosine residues present within the dinucleotide CpG. Treatment of DNA with bisulfite converts unmodified cytosines to uracil, whereas 5-methylcytosine residues are unaffected. As a consequence, treatment with bisulfite introduces specific sequence changes in DNA molecules that result from the methylation statuses of cytosine residues present therein. Sequencing of nucleic acids that have been treated with bisulfite (i.e., “bisulfite sequencing”) can then be used to determine the overall methylation status of the nucleic acid by comparing the sequence identified with a standard sequence (i.e., the same nucleic acid sequenced without bisulfite treatment).

Other strategies can also be employed to determine the methylation patterns at loci of interest subsequent to bisulfite treatment. Exemplary such methods include restriction analysis using endonucleases that differentially restrict DNA based on differences in methylation (see e.g., Sadri et al. (1996) Nucleic Acids Res (1996) 24:4987-4989).

Another technique that can be employed to identify the methylation status of a nucleic acid is the and combined bisulfite-restriction analysis (COBRA) technique (Xiong & Laird-(1997) Nucleic Acids Res 25:2532-2534). In this method, standard bisulfite treatment is used to introduce methylation-dependent sequence differences into a nucleic acid (for example, a subsequence of a genomic DNA). The nucleic acid (or a subsequence thereof) is then PCR amplified using primers that flank the sequence to be assayed. The bisulfite treatment results in the PCR amplification products having sequences that reflect the presence or absence of methylated-cytosines in the original nucleic acid molecule. Any sequence changes that result can lead to the methylation-dependent creation of new restriction enzyme sites or it can lead to the methylation-dependent retention of pre-existing sites such as. The products of the PCR reaction are then digested with appropriate restriction enzymes, and the products of the digestion reactions are visualized. Based on the sizes of the digestion products, it is possible to determine the methylation statuses of known sequences presented in the original nucleic acid molecule.

Carrier Chromatin-Immunoprecipitation (Carrier-ChIP; O'Neill et al. (2006) Nat Genet 38:835-841) can also be employed to assay DNA methylation. A kit for performing this assay is commercially available (Magna ChIP™ G kit, Upstate-Millipore, Billerica, Mass., United States of America).

In certain embodiments, the biological age measure is the level of expression of one or more of Oct4, Nanog, Sox2, Klf4, or cMyc in VSELs. Methods for determining expression of these genes and proteins are known and well within the ability of one of skill in the art. Furthermore, oligonucleotides, antibodies, and other reagents are commercially available.

Using any of these exemplary techniques, either alone or in combination, the methylation status of different cell preparations (e.g., preparations of VSELs or other cell types of interest including, but not limited to other types of stem cells) can be determined. After methylation statuses are determined, they can be compared to identify how they differ among different cell types (e.g., stem cell types). For example, the methylation statuses of various loci of exemplary totipotent cells such as ES cells can be compared to the methylation statuses of the same loci in more differentiated (i.e., less pluripotent) cells such as HSCs, bone marrow mononuclear cells (BMMNCs), and/or MSCs. Given the relative levels of pluripotency of these cell lines, methylation profiles for these cell types can be established and compared to the methylation profiles of cell types of interest such as, but not limited to VSELs.

The invention also provides a method for deciding whether treatment or intervention that impedes progression of an aging-related VSEL characteristic is appropriate. In one embodiment, treatment is appropriate when the biological age of a subject is determined to be greater than its chronological age. In one embodiment, treatment or intervention is appropriate when the biological age of a subject exceeds its chronological age by one standard deviation or more. In another embodiment, treatment or intervention is appropriate when the biological age of a subject exceeds its chronological age by two or more standard deviations.

In certain embodiments, treatment to reduce or impede the increase of biological age is elective. In such embodiments, the biological age of a subject can be less than, equal to, or greater than its chronological age. In one such embodiment, the invention provides a method of establishing a baseline biological age prior to treatment. In another such embodiment, the invention provides a method for monitoring treatment-responsive changes in biological age. Accordingly, the method is applied after a subject has been treated, for example, by caloric restriction, regular exercise, or with an agent that reduces insulin/insulin-like growth factor signaling.

In one approach, treatment of biological age involves reducing insulin/insulin-like growth factor signaling, for example by modulating the activity of IGF-R, PIK3, mTOR, AKT, RasGRF-1, or other components of the pathway. Inhibitors of these cellular components include biological agents, such as antibodies, as well as chemical agents. The term “chemical agent” refers to a substances that has a molecular weight up to 2000 atomic mass units (Daltons). Such substances are sometimes referred to as “small molecules.” As used herein, “biological agents” are molecules which include proteins, polypeptides, and nucleic acids, and have molecular weights equal to or greater than 2000 atomic mass units (Daltons). Several such compounds have been developed and are known in the art, such as antibodies that bind to IGF-R and block receptor activation, and inhibitors of mTOR, such as, but not limited to rapamycin. Other compounds that are direct or indirect inhibitors include metformin and PPAR-gamma receptor antagonists.

In another embodiment, an inhibitor of growth hormone receptor is administered. Elevated growth hormone levels increase IGF-1 in plasma, which is detrimental to VSELs. Also, VSELs express growth hormone receptor at high levels. An example of a growth hormone antagonist is Pegvisomant (B2036), which is a pegylated GH analog.

In another embodiment, an agent that is a natural product, or “nutriceutical” is administered. Lycopene is one such compound, and is an IGF-1 signal transduction inhibitor. Another such compound is resveratrol, which is reported to suppress cell proliferation by inhibiting IGF-1R and its downstream signaling pathway (i.e., suppression of IGF-1R/Akt/Wnt signaling). Epigallocatechin gallate (ECGC) and curcumin are reported to inhibit mTOR.

In another embodiment, stem cells are administered to the subject. The stem cells may be from a different donor and matched to the subject. Preferably the stem cells are autologous. Autologous preparations of stem cells may be collected from a subject and stored for later infusion. The stem cells may have been collected from the subject at an earlier time, for example from cord blood, or peripheral blood. In a preferred embodiment, the stem cells were collected by an apheresis procedure and mononuclear cells stored. In a preferred embodiment, the stem cells were mobilized at the time of collection to increase their number. In another preferred embodiment, the stem cell preparation was enriched for one or more stem cell types, including, but not limited to VSELs and MSCs. Having been collected at an earlier age and stored, the stem cells, when infused, have undergone limited or no age-related loss of self-renewal ability or pluripotency.

It is to be understood and expected that variations in the principles of invention herein disclosed may be made by one skilled in the art and it is intended that such modifications are to be included within the scope of the present invention.

Throughout this application, various publications are referenced. These publications are hereby incorporated into this application by reference in their entireties to more fully describe the state of the art to which this invention pertains. The following examples further illustrate the invention, but should not be construed to limit the scope of the invention in any way.

### EXAMPLES

The following examples provide illustrative embodiments. In light of the present disclosure and the general level of skill in the art, those of skill will appreciate that the following Examples are intended to be exemplary only, and that numerous changes, modification, and alterations can be employed without departing from the scope of the presently disclosed subject matter.

### Example 1

VSELs and their pluripotency decrease during aging.

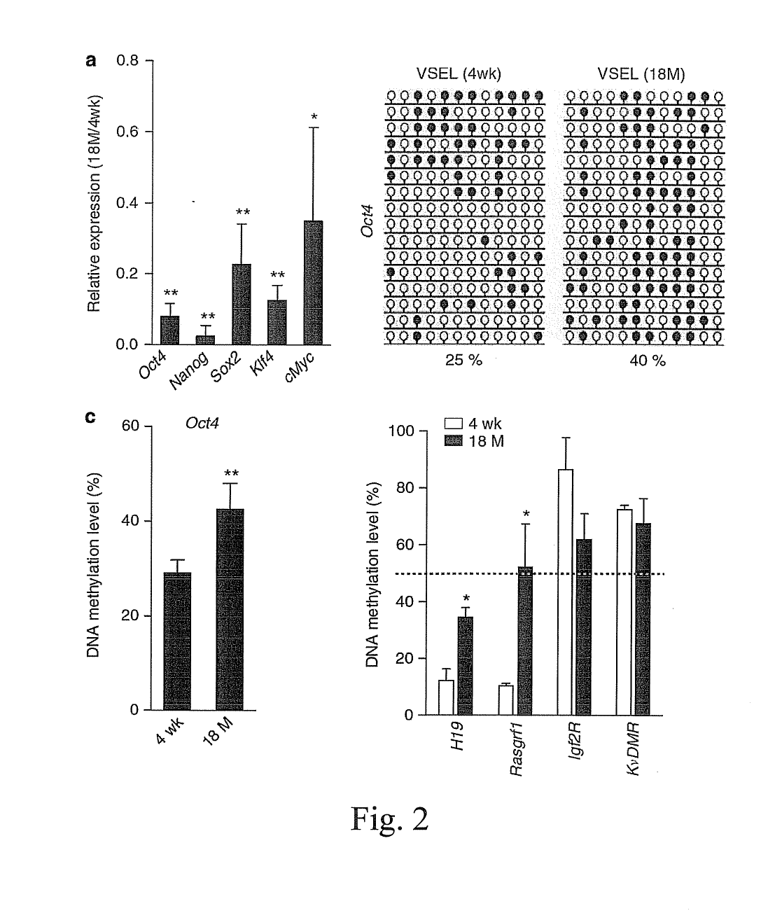

Studies performed on normal young (4-week-old) and old (2-year-old) mice revealed that the number of VSELs and their pluripotentiality decreased during ageing. Accordingly, VSELs from old mice show lower expression of the pluripotentiality master-regulators, such as Oct4, Nanog, Sox2, Klf4 and cMyc (FIG. 2a) and, at the molecular level, the Oct4 promoter in VSELs becomes hypermethylated with age and shows a closed chromatin structure (FIG. 2b). Furthermore, VSELs from old mice show the somatic type of methylation at both Igf2-H19 and Rasgrf1 loci, which suggests that VSELs from old mice have increased sensitivity to Ins factor signaling (FIG. 2c).

### Example 2

Chronic Insulin/Insulin-like Growth Factor Signaling Accelerates the Depletion of VSELs.

Laron dwarf mice have a deficiency of GH receptor and display a severe reduction in the Igf1 plasma level, and do not display an increase in GET-mediated Igf-1 plasma level in response to caloric uptake. These animals live 30-40% longer than their normal littermates. The number of VSELs in BM of 8-month and 20-month-old murine Laron dwarfs (GHR^{-/-}) and corresponding normal heterozygous littermates (GHR^{+/-}) were measured by FACS. The number of VSELs in the BM of plasma Igf1-deficient Laron dwarfs is maintained at a three- to four-fold higher level than normal GHR^{+/-} littermates during aging (FIG. 3a). Molecular analysis studies additionally demonstrate that the Oct4 promoter in these animals shows also a higher level of demethylation (FIG. 3b). While analysis of peripheral blood cell counts did not reveal any differences in the number of erythrocytes, platelets and leukocytes between Laron dwarf mice and wild-type controls, and all these mice have normal histology of bone marrow tissue (data not shown), we observed that Laron dwarf mice have in BM: (i) a approximately threefold increase in the number of Sca-1⁺c-kit⁺lin hematopoietic SCs (8-month olds) and (ii) up to fourfold higher number of clonogenic CFU-GM, BFU-E and CFU-Meg cells (8- and 20-month old) (FIG. 3c).

For the following Examples 3-5, the following methods and conditions were used.

Treatment of Mice with Porcine GH or Human IGF-I

Young Ames dwarf mice (Prop ^{df/df})—Groups of 10 male Ames dwarf and WT males were subjected to treatment with porcine GH (pGH) via subcutaneous (sc) injection (6 fig/g/day), given twice daily starting at the age of 2 weeks and continuing for 6 weeks. On Saturdays and Sundays animals were injected only once with a full dosage following a previous protocol (Masternak MM et al., 2010). Control Ames dwarfs (df/df) and wild type (WT) littermates of the same age were treated with saline. After 6 weeks of pGH treatment half of the animals were sacrificed and tissues and cells were collected for immediate analysis. The other half of the animals had pGH treatment discontinued and the animals were kept untreated for 12 weeks before final analysis.

Adult Ames dwarf mice (Prop ^{df/df})—Groups of 9-10 male Ames dwarf and WT males were subjected to treatment with porcine GH (pGH) via subcutaneous (Sc) injection (6 μg/g/day), given twice daily starting at the age of 6 months and continuing for 6-8 weeks. On

Saturdays and Sundays animals were injected only once with a full dosage following a previous protocol. Control male Ames dwarfs (df/df) and wild type (WT) male littermates of the same age were treated with saline.

Laron dwarf GHR^{-/-} mice—Young male GHRKO mice were subjected to human IGF-I (Biovision Inc.) treatment via subcutaneous (sc) injection (4 μg/g/day), given twice daily starting at the age of 6 weeks and continuing for 3 weeks. Control male GHRKO and wild type (WT) male littermates of the same age were treated with saline.

Peripheral Blood Parameter Counts

Fifty microliters of PB was taken from the retro-orbital plexus of the mice and collected into microvette EDTA-coated tubes (Sarstedt Inc., Newton, N.C.). Samples were run within 2 hours of collection on a Hemavet 950 (Drew Scientific Inc., Oxford, Conn.).

Staining and Sorting of BM-Derived Stem Cells

To determine the amount of Sca-1 ⁺Lin⁻CD45⁻ (VSEL) and Sca-1⁺Lin⁻CD45⁺ (HSC) cells, flow cytometry analysis was perfomed. In brief, a single cell suspension was stained for lineage markers (CD45R/B220 clone RA3-6B2, Gr-1 clone RB6-8C5, TCRαβ clone H57-597, TCRK clone GL3, CD11b clone M1/70, and Ter-119 clone TER-119) conjugated with phycoerythrin (PE), CD45 (clone 30-F11) conjugated with APC-Cy7, and Sca-1 (clone D7) conjugated with PECy5 for 30 minutes on ice. After washing, samples were analyzed by fluorescence-activated cell sorting (LSRII) (BD Biosciences). At least 10⁶ events were acquired and analyzed by using BD FACSDiva™ software. Samples stained with appropriate isotype controls (BD Pharmingen, San Diego, Calif.) were examined in parallel.

Isolation and Fluorescence-Activated Cell Sorting (FACS) of VSELs and HSC from Murine BM

VSELs and HSC were isolated from BM of adult female or male mice. Briefly, BM was flushed from tibias and femurs and the population of total nucleated cells (TNCs) was obtained after lysis of red blood cells using 1×BD Pharm Lyse Buffer (BD Pharmingen, San Jose, Calif., USA). TNCs were subsequently stained for CD45, hematopoietic lineage markers (Lineage [Lin]), and Sca-1 antigen for 30 minutes in medium containing 2% fetal bovine serum. The following anti-mouse antibodies (BD Pharmingen) were used for staining: rat anti-CD45 (allophycocyanin-Cy7; clone 30-F11), anti-CD45R/B220 (phycoerythrin [PE]; clone RA3-6B2), antiGr-1 (PE; clone RB6-8C5), antiT-cell receptor-αβ (PE; clone H57-597), antiT-cell receptor-γδ (PE; clone GL3), anti-CD11b (PE; clone M1/70), anti-Ten 19 (PE; clone TER-119), and anti-Ly-6A/E (Sca-1) (biotin; clone E13-161.7, with streptavidin-conjugated to PE-Cy5). Cells were then washed, re-suspended in RPMI 1640 medium with 2% fetal bovine serum, and sorted by MoFlo cell sorter (Dako, Carpinteria, Calif., USA). The Sca-1 ⁺Lin⁻CD45⁻ cells (VSELs) and control Sca-1 ⁺Lin⁻CD45⁺ cells (HSCs) were isolated.

Bisulfite-Sequencing (BSS) and Combined Bisulfite-Restriction Analysis (COBRA)

The DNA methylation status of the DMRs of Igf2-H19 and RasGRF1 loci were investigated using bisulfite DNA modification followed by sequencing as well as by COBRA assay. In brief, genomic DNA for VSELs, HSCs, and BMMNCs isolated from the indicated mice strains were prepared using the DNeasy Blood & Tissue Kit (Qiagen Inc, Valencia, Calif., USA). Next, 100 ng of gDNA were used in bisulfite modification, performed using the EpiTect Bisulfate Kit (Qiagen Inc) according to the manufacturer's instructions. The BSS and COBRA analysis were performed as previously described (Shin DM et al., 2009).

### Example 3

Increase in Plasma Circulating Insulin-Like Growth Factor-1 (IGF-I) Level Leads to Premature Depletion of VSELs from murine BM.

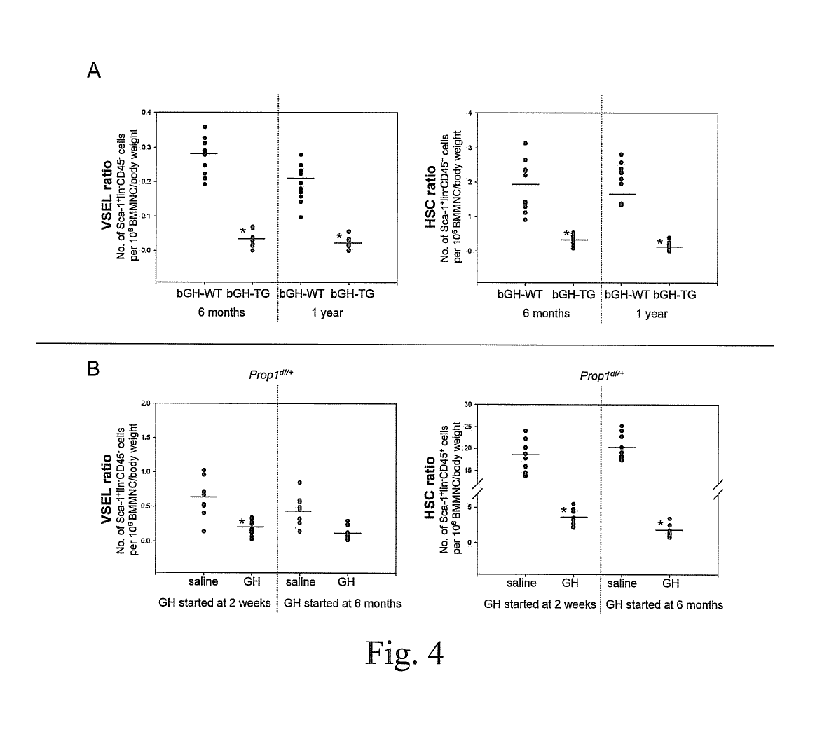

The number of VSELs in BM of transgenic mice that overexpress bovine growth hormone (bGH) under control of phosphoenolpuryvate carboxykinase (PEPCK) promoter. These mice live ˜1 year, ˜50% shorter than control littermates that lack this transgene. At 12 months of age these animals have even more severely reduced numbers of Sca-1⁺ lin⁻CD45⁻ VSELs and Sca-1⁺ lin⁻ CD45⁺ HSCs in BM. In contrast, control mice maintain number of HSCs in BM, however they have lower number of VSELs at age of 12 months. (FIG. 4A).

Prolonged increase in plasma GH levels by twice-daily injections of porcine GH has a similar effect. 2 week and 6 month old wild type (wt) mice were divided in two groups and were treated for 8 weeks with pGH (6 μg/g/day) or saline (control group). Like the bGH transgenic mice, both 2 week and 6 month old wt animals injected with pGH for 8 weeks exhibited reduction in number of VSELs and HSCs in BM. (FIG. 4B). These results are consistent with GH stimulation of IGF-I secretion from liver, leading to high levels of circulating IGF-I in bGH transgenic and wt mice injected with pGH.

### Example 4

Long living Laron (GHR^{-/-}) and Ames (Prop1^{df/df}) Dwarf Mice with Low Circulating Level of IGF-I Retain High Number of VSELs in BM During Aging.

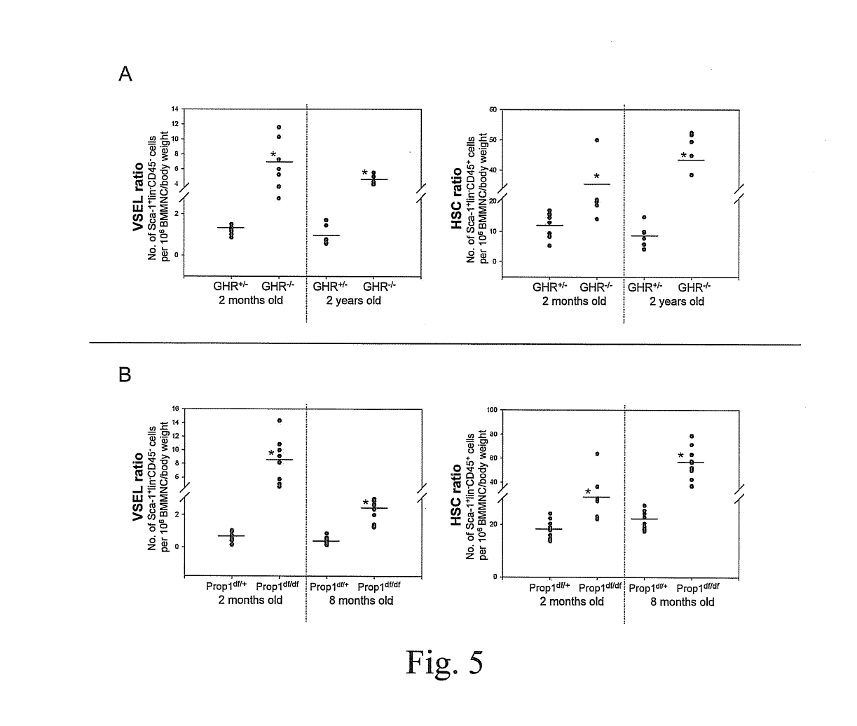

The number of VSELs and HSCs in 2 year old Laron dwarf mice (GHR^{-/-} and in a different type of long-lived mutant mice with low plasma circulating IGF-I, 8 month old Ames dwarfs (Prop1^{df/df}) were compared with normal control littermates GHR^{-/+} and Prop1^{df/+} animals. Both murine mutants exhibit extended longevity and have low level of circulating IGF-I, but the basis for these phenotypic characteristics is different. While, Laron dwarf mice (GHR^{-/-}) have a deletion of GH-receptor (GHR) that makes them insensitive to GH signaling and thus they do not secrete IGF-I from liver, the Ames dwarfs (Prop1^{df/df}) have a mutation of prophet of pituitary factor 1 (Prop 1) gene involved in regulation of pituitary specific transcription factor 1 which leads to a combined defect in expression of GH, Thyroid-stimulating hormone (TSH) and prolactin (PRL). Both Laron dwarfs and Ames dwarfs have higher number of VSELs and HSCs in BM as compared to age matched normal littermates (GHR^{-/+} and Prop1^{df/+} (FIG. 5A and B, respectively).

### Example 5

Short-Term GH or IGF-I Treatment Reduces the Number of VSELs and HSCs in Low Circulating Plasma IGF-I Level, Long living Ames (Prop1^{df/df}) and Laron (GHR^{-/-}) dwarf mice.

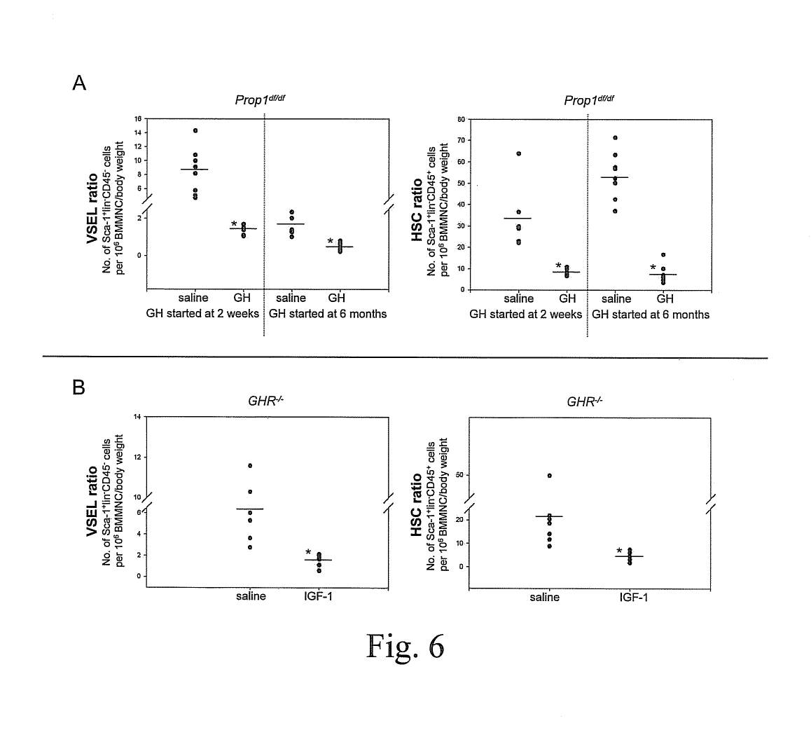

Ames and Laron dwarf mice were treated with pGH or recombinant human IGF-I injections, respectively. As is it shown in FIG. 6A, Ames dwarfs (Prop1^{df/df}) treated for 6 weeks starting at the age of 14 days or 6 months by twice-daily injection of pGH (6 μg/g/day) exhibit significantly reduced number of VSELs and HSCs in BM as compared to Ames dwarfs injected with saline vehicle (controls). These mice have low circulating levels of IGF-I due to GH deficiency; injections of GH elevated their circulating IGF-I levels.

To confirm a direct role of IGF-I, low circulating plasma IGF-I level Laron dwarf mice (6 weeks of age) were injected twice-daily for 3 weeks with 2 μg/g/day human recombinant IGF-I (hIGF-I). As shown in FIG. 6B, hIGF-I injection led to a significant decrease in number of VSELs and HSCs in these animals as compared to control Laron dwarf mice injected with saline.

### Example 6

Changes in Methylation Pattern of Imprinted Genes Involved in Ins/Igf Signaling.

VSELs deposited in adult BM are in a quiescent state protected from proliferation by erasure of differently methylated regions (DMRs) in paternally imprinted genes that encode Igf2-H19 and RasGRF1. These cells may be prematurely depleted by chronic IIS signaling. Thus changes in expression of imprinted genes in VSELs lead to downregulation of i) IGF-2 that is an autocrine factor involved in proliferation of VSELs and ii) RasGRF1 a GTP exchange factor (GEF) for signaling from InsR and IGF-IR. These both changes play a major role in attenuating IIS in these cells (Ratajczak M Z et al., 2011).

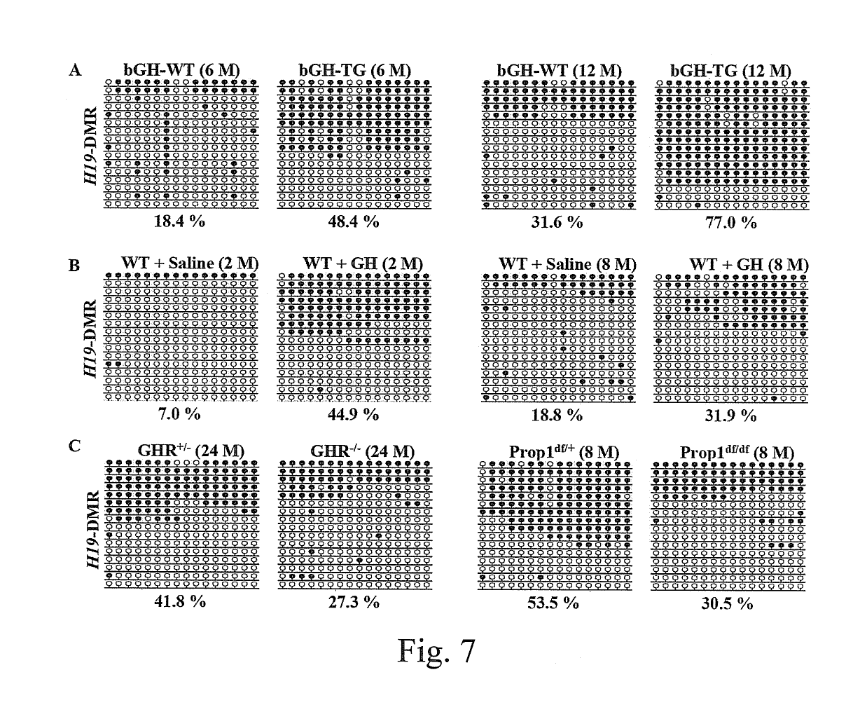

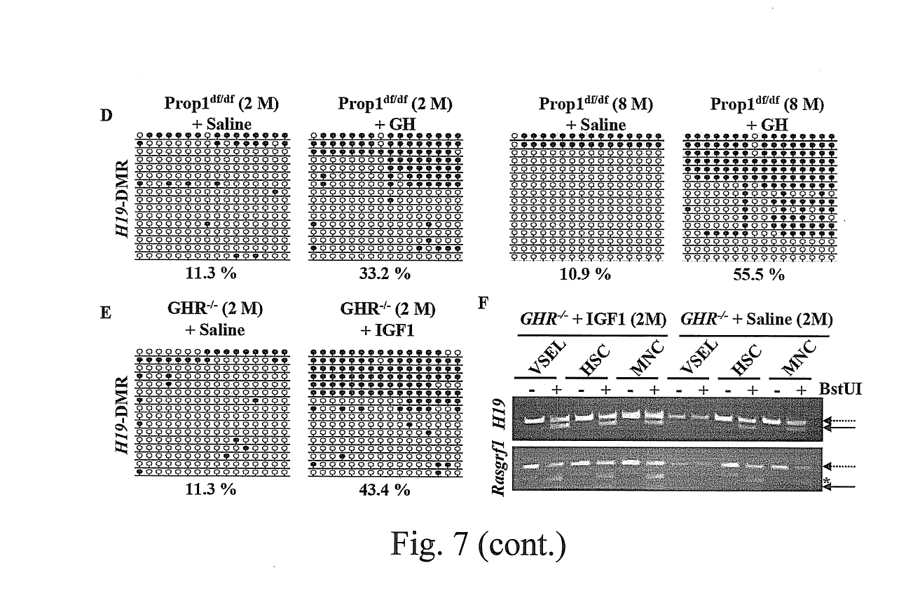

To address the status of paternal imprints in VSELs, imprinting (methylation) of DMRs was evaluated in Igf2-H19 and RasGRF1 loci by employing bisulfide modification of DNA followed by sequencing (FIG. 7) or by employing restriction analysis of PCR products employing primers designed around DMRs of interest (COBRE assay). In wild type young mice, the DNA of these paternally imprinted regions in VSELs, in contrast to HSCs and BMMNCs remain unmethylated (FIG. 7). Furthermore, decrease in pool of VSELs after chronic exposure to GH/IGF-I (FIG. 2), corresponded with enhanced methylation of DMR for Igf2-H19 locus in VSELs as seen in both bGH transgenic mice (FIG. 7A) and wild type mice injected with pGH (FIG. 7B).

Erasure of genomic imprints on Igf2-H19 in VSELs turns during ageing into normal somatic pattern. VSELs from 2 year old wild type or Laron dwarf heterozygote mice (GHR^{-/+}) and 8 months old control mice for Ames dwarfs (Prop1^{df/+}) show the somatic type (˜50% of methylation) on DMR for Igf2-H19 locus (FIG. 7C). In contrast, VSELs from both Laron dwarf (GHR^{-/-}) and Ames (Prop1^{df/df}) mice exhibit delay in ageing related DNA methylation of Igf2-H19 locus (˜30% of methylation) (FIG. 7C). This locus became prematurely methylated if young 2 month old Ames- or 8 month old Laron-dwarf mice were exposed to daily pGH and IGF-I injections (FIG. 7D-F).

Taken together, these results suggest the prolonged IIS signaling can induce reprogramming of the DNA methylation of imprinted loci (Igf2-H19 and RasGRF1) and increase sensitivity of these cells to IIS, potentially leading to premature depletion of primitive VSELs from adult BM.

### Example 7

Biological Age and Aging Rate of a Test Subject

Blood samples from 50 subjects aged 25 years ±6 months, 50 subjects aged 50 years ±6 months, and 50 subjects aged 65 years ±6 months are tested to determine both the number of VSELs and the proportion total nucleated cells (TNCs) that are VSELs, in 10 ml of blood. To measure VSELs, the blood sample is contacted with a panel of labeled antibodies that bind to CD45, CD34, CD133, CXCR4, and at least one lineage marker (e.g., CD41). The cells that are CD45⁻/lin⁻ and express at least one of CD34, CXCR4, and CD133 (e.g., CXCR4) are counted. The data is used to plot a standard curve showing the proportion (p) of TNCs that are VSELs as a function of age. The distribution of p for each age group is calculated to provide a measure of the variability of p among the samples.

The proportion p of VSELs in a blood sample from a test subject is measured and compared to the standard curve, and a biological age, with an error value derived from the variability of p, is assigned, and the subject is informed of his or her approximate biological age.

The test subject may be retested at a later time, for example to determine a biological aging rate or measure the effect of an intervening treatment or regimen intended to reduce the biological aging rate or restore VSEL number or function.

1. A method of determining a mammal's biological age relative to a reference chronological age for the mammal, which comprises:
(a) obtaining a tissue or blood sample from the mammal;
(b) determining the number of VSELs in the tissue or blood sample;
(c) comparing the number of VSELs determined in step (b) to a reference number of VSELs which represents the reference chronological age for mammals of the species;
thereby determining the relative biological age of the subject mammal.

2. The method of claim 1, wherein the reference number of VSELs is determined from the number of VSELs in a similar tissue or blood sample from a population of mammals of the same species and chronological age.

3. The method of claim 1, wherein the reference number of VSELs is determined from an age-dependent profile for a population of mammals of the same species having different chronological ages.

4. The method of claim 1, wherein the number of VSELs is expressed as a percent of total nucleated cells.

5. The method of claim 1, wherein the number of VSELs is determined by measuring the number of cells in the tissue or blood sample that have a preselected pattern of markers.

6. The method of claim 5, wherein the markers are measured using antibodies.

7. The method of claim 5, wherein the preselected pattern of markers is CD34⁺, lin⁻, CD45⁻.

8. The method of claim 7, wherein the preselected pattern of markers further comprises at least one of CD133⁺ and CXCR4⁺.

9. The method of claim 7, wherein the cells express at least one of SSEA-4, Oct- 4, Rex-1, and Nanog.

10. The method of claim 5, wherein the measured cells in the tissue or blood sample have a diameter of 4-5 μm, 4-6 μm, 4-7 μm, 5-6 μm, 5-8 μm, 6-9 μm, or 7-10 μm.

11. The method of claim 5, wherein the measured cells in the tissue or blood sample possess large nuclei surrounded by a narrow rim of cytoplasm.

12. The method of claim 1, wherein the tissue or blood sample is bone marrow.

13. The method of claim 1, wherein the tissue or blood sample is peripheral blood.

14. The method of claim 13, which further comprises administering an amount of an agent that mobilizes VSELs before obtaining the blood sample.

15. The method of claim 14, wherein the agent that mobilizes VSELs is G-CSF.

16. The method of claim 1, wherein the mammal is a human.

17. The method of claim 1, wherein if the number of VSELs determined in step (b) indicates a relative biological age greater than the chronological age, the mammal is treated to reduce insulin/insulin-like growth factor signal transduction.

18. The method of claim 1, wherein prior to step (a), an agent that inhibits insulin/insulin like growth factor signal transduction is administered to the mammal.

19. The method of claim 17, wherein the mammal is treated with a compound that inhibits signal transduction by one or more of IGF-R, PI3K, mTOR, AKT, and RasGRF-1.

20. The method of claim 19, wherein the compound is a chemical agent.

21. The method of claim 19, wherein the compound is a biological agent.

22. The method of claim 1, wherein if the number of VSELs determined in step (b) indicates a relative biological age greater than the chronological age, the mammal is treated with resveratrol.

23. The method of claim 1, wherein if the number of VSELs determined in step (b) indicates a relative biological age greater than the chronological age, the mammal is treated with lycopene.

24. The method of claim 1, wherein if the number of VSELs determined in step (b) indicates a relative biological age greater than the chronological age, the mammal is administered a low calorie diet.

25. The method of claim 1, wherein if the number of VSELs determined in step (b) indicates a relative biological age greater than the chronological age, VSELs are administered to the mammal.

26. The method of claim 25, wherein the administered VSELs are autologous VSELs.

27. The method of claim 26, wherein the administered VSELs were collected from the mammal at an earlier time and stored.

28. The method of claim 25, wherein the administered VSELs express selected markers of pluripotency at a higher level than VSELs of the obtained tissue or blood sample.

29. The method of claim 28, wherein there markers of pluripotency include one or more markers selected from Oct4, Nanog, Sox2, Klf4, and cMyc.

30. A method of determining a mammal's biological age relative to a reference chronological age for the mammal, which comprises:
(a) obtaining a tissue or blood sample from the mammal;
(b) determining the methylation state of one or more of the Oct4 promoter, the Igf2-H19 locus, and the RasGRF-1 locus of VSELs in the tissue or blood sample;
(c) comparing the methylation state determined in step (b) to a reference methylation state of the one or more of the Oct4 promoter, the Igf2-H19 locus, and the RasGRF-1 locus of VSELs which represent the reference chronological age for mammals of the species;
thereby determining the relative biological age of the subject mammal.

31-56. (canceled)

57. A method of determining a mammal's biological age relative to a reference chronological age for the mammal, which comprises:
(a) obtaining a tissue or blood sample from the mammal;
(b) determining the level of expression of one or more of Oct4, Nanog, Sox2, Klf4, and cMyc in VSELs in the tissue or blood sample;
(c) comparing the level of expression determined in step (b) to a reference level of expression of one or more of Oct4, Nanog, Sox2, Klf4, and cMyc in VSELs which represents the reference chronological age for mammals of the species; thereby determining the relative biological age of the subject mammal.

58-83. (canceled)
